(19) United States
(12) Patent Application Publication
(10) Pub. No.: US 2024/0277422 A1
(43) Pub. Date: Aug. 22, 2024

(54) THROMBECTOMY SYSTEM USER INTERFACE

(71) Applicant: Boston Scientific Scimed, Inc., Maple Grove, MN (US)

(72) Inventors: Brenton Smith, Minneapolis, MN (US); Heather Moulton, White Bear Lake, MN (US); Mark Alfred Hilse, Somerset, WI (US); Heidi Robertson, Eden Prairie, MN (US); Jeffry V. Marshik, New Brighton, MN (US); Pranam Shetty, Plymouth, MN (US); Alisa Rantanen, Chicago, IL (US)

(73) Assignee: Boston Scientific Scimed, Inc., Maple Grove, MN (US)

(21) Appl. No.: 18/441,298

(22) Filed: Feb. 14, 2024

Related U.S. Application Data
(60) Provisional application No. 63/446,150, filed on Feb. 16, 2023.

(51) Int. Cl. A61B 34/00; A61B 17/22

(52) U.S. Cl. CPC A61B 34/25; A61B 17/22; A61B 2017/22079

(57) ABSTRACT

A user interface for a thrombectomy fluid management system may include a first mode indicator configured to indicate when a first mode of fluid inflow is active, a second mode indicator configured to indicate when a second mode of fluid inflow is active, and a run time display including an alphanumeric run time value shown in a first color. The alphanumeric run time value is a sum of a first run time value of the first mode and a second run time value of the second mode. The system may include an inflow pump configured to provide fluid inflow through a thrombectomy device, a controller in communication with the inflow pump and the thrombectomy device, a user activation switch configured to activate the inflow pump, and a display including the user interface. The display may include a touchscreen user interface.

### CROSS-REFERENCE TO RELATED APPLICATIONS

This application claims the benefit of U.S. Provisional Patent Application Ser. No. 63/446,150, filed Feb. 16, 2023, which is incorporated herein by reference.

### TECHNICAL FIELD

The disclosure relates generally to thrombectomy systems. More particularly, the disclosure relates to user interfaces for thrombectomy systems.

### BACKGROUND

Thrombectomy is a procedure for removing thrombus, plaques, lesions, clots, etc. from the vasculature of a patient. Mechanical and fluid-based systems can be used to remove thrombus. With fluid-based systems, an infusion fluid (e.g., saline) may be infused to a treatment area of a vessel with a catheter to soften, loosen, and/or dislodge the thrombus. In some instances, an effluent (e.g., the infusion fluid and/or blood) including the dislodged thrombus may be extracted from the vessel through the catheter. Of the known thrombectomy systems and methods, there is an ongoing need to provide alternative configurations of thrombectomy catheters and systems, as well as methods of operating such thrombectomy systems.

### SUMMARY

In one example, a user interface for a thrombectomy fluid management system, may comprise: a first mode indicator configured to indicate when a first mode of fluid inflow is active; a second mode indicator configured to indicate when a second mode of fluid inflow is active; and a run time display including an alphanumeric run time value shown in a first color. The alphanumeric run time value may be a sum of a first run time value of the first mode and a second run time value of the second mode.

In addition or alternatively to any example disclosed herein, the run time display shows a first recommended run time threshold corresponding to a first working condition and a second recommended run time threshold corresponding to a second working condition.

In addition or alternatively to any example disclosed herein, the alphanumeric run time value is configured to change from the first color to a second color when the sum reaches at least one of the first and second recommended run time thresholds.

In addition or alternatively to any example disclosed herein, the alphanumeric run time value is configured to change from the first color to a second color when the sum reaches the first recommended run time threshold.

In addition or alternatively to any example disclosed herein, the alphanumeric run time value is configured to change from the first color to a second color when the sum reaches the second recommended run time threshold.

In addition or alternatively to any example disclosed herein, the alphanumeric run time value is configured to change to a third color when the sum reaches the second recommended run time threshold.

In addition or alternatively to any example disclosed herein, the first mode indicator is user selectable to activate the first mode of fluid inflow.

In addition or alternatively to any example disclosed herein, the second mode indicator is user selectable to activate the second mode of fluid inflow.

In addition or alternatively to any example disclosed herein, the alphanumeric run time value is shown in seconds.

In addition or alternatively to any example disclosed herein, a thrombectomy fluid management system may comprise: an inflow pump configured to provide fluid inflow through a thrombectomy device; a controller in communication with the inflow pump and the thrombectomy device; a user activation switch configured to activate the inflow pump; and a display including a user interface. The user interface may include a first mode indicator configured to indicate when a first mode of fluid inflow is active; a second mode indicator configured to indicate when a second mode of fluid inflow is active; and a run time display including an alphanumeric run time value shown in a first color. The alphanumeric run time value may be a sum of a first run time value of the first mode and a second run time value of the second mode.

In addition or alternatively to any example disclosed herein, the run time display shows a first recommended run time threshold corresponding to a first working condition and a second recommended run time threshold corresponding to a second working condition.

In addition or alternatively to any example disclosed herein, the alphanumeric run time value is configured to change from the first color to a second color when the sum reaches at least one of the first and second recommended run time thresholds.

In addition or alternatively to any example disclosed herein, the alphanumeric run time value is configured to change from the first color to a second color when the sum reaches the first recommended run time threshold.

In addition or alternatively to any example disclosed herein, the alphanumeric run time value is configured to change from the first color to a second color when the sum reaches the second recommended run time threshold.

In addition or alternatively to any example disclosed herein, the controller is configured to emit a first audible notification when the sum reaches the first recommended run time threshold.

In addition or alternatively to any example disclosed herein, the alphanumeric run time value is configured to change to a third color when the sum reaches the second recommended run time threshold.

In addition or alternatively to any example disclosed herein, the controller is configured to emit a second audible notification when the sum reaches the second recommended run time threshold.

In addition or alternatively to any example disclosed herein, after reaching the first recommended run time threshold, the inflow pump continues to run whenever the user activation switch is activated.

In addition or alternatively to any example disclosed herein, after reaching the second recommended run time threshold, the inflow pump continues to run whenever the user activation switch is activated.

In addition or alternatively to any example disclosed herein, a thrombectomy fluid management system may comprise: an inflow pump configured to provide fluid inflow through a thrombectomy device; a controller in communication with the inflow pump and the thrombectomy device; a user activation switch configured to activate the inflow pump; and a display including a touchscreen user interface. The touchscreen user interface may include a first mode indicator configured to highlight when a first mode of fluid inflow is active, the first mode indicator being user selectable on the touchscreen user interface; a second mode indicator configured to highlight when a second mode of fluid inflow is active, the second mode indicator being user selectable on the touchscreen user interface; and a run time display including an alphanumeric run time value shown in a first color when fluid inflow is started. The alphanumeric run time value may be a sum of a first elapsed run time of the inflow pump when activated in the first mode and a second elapsed run time of the inflow pump when activated in the second mode.

In addition or alternatively to any example disclosed herein, the run time display shows a first recommended run time threshold corresponding to a first working condition and a second recommended run time threshold corresponding to a second working condition.

In addition or alternatively to any example disclosed herein, the controller includes data acquisition device configured to communicate with the thrombectomy device to obtain specifications associated with the thrombectomy device.

In addition or alternatively to any example disclosed herein, the specifications include the first recommended run time threshold and the second recommended run time threshold.

In addition or alternatively to any example disclosed herein, the touchscreen user interface includes a graphical display adjacent the alphanumeric run time value showing the alphanumeric run time value relative to the first recommended run time threshold and the second recommended run time threshold.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a perspective view of a thrombectomy system;

FIG. 2 is a partially exploded perspective view of a portion of the system shown in FIG. 1;

FIG. 3-6 illustrate selected aspects of a user interface for a thrombectomy system; and

FIG. 7 illustrates selected aspects of the user interface and a look-up table for selected specifications associated with different thrombectomy devices.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

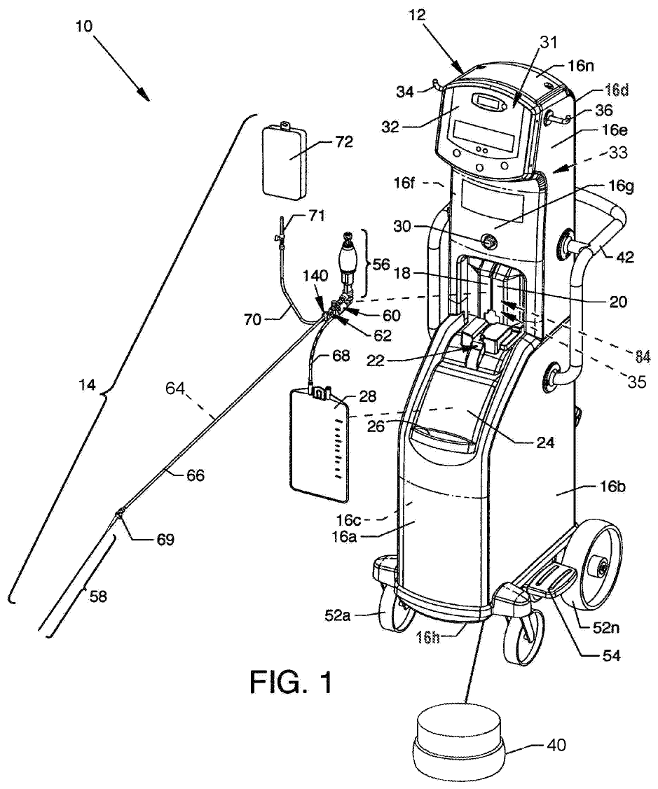

FIG. 1 is a perspective view of an illustrative thrombectomy system 10. The thrombectomy system 10 may include a drive unit 12 and a pump/catheter assembly 14. In some embodiments, the drive unit 12 may include or may be referred to as a control console. In some instances, the pump/catheter assembly 14 may be a disposable single use device in which a new pump/catheter assembly 14 may be used with the drive unit 12 for each medical procedure. Shown on the drive unit 12 are a plurality of removable panels 16a-16n about and along the drive unit 12 enclosing the internal structure of the drive unit 12. An illustrative drive unit 12 is described in commonly assigned U.S. Pat. No. 7,935,077, titled THROMBECTOMY CATHETER DEPLOYMENT SYSTEM, the disclosure of which is hereby incorporated by reference. Centrally located in the drive unit 12 and aligned to the lower region of the panel 16g may be doors 18 and 20 which open to expose an interior of the drive unit 12 to provide access to a carriage assembly 22. The carriage assembly 22, which may accommodate components of the pump/catheter assembly 14, as discussed further herein, is accessible via opening the doors 18 and 20. In some embodiments, the doors 18 and 20 may be configured to open automatically. In some embodiments, the doors 18 and 20 may be configured to open in response to movement or activation of the carriage assembly 22. In some embodiments, the drive unit 12 may include a catch basin for collecting fluid leakage from the components of the pump/catheter assembly 14. In one non-limiting example, a removable drip tray 24 may be located on the front of the drive unit 12 extending from below the carriage assembly 22 toward the panel 16a. Other configurations of catch basins are also contemplated. The removable drip tray 24 and a removable receptacle 26 may collectively support and accommodate an effluent collection bag, such as effluent collection bag 28 of the pump/catheter assembly 14. In some embodiments, the drive unit 12 may include a different structure, such as a hook for hanging the effluent collection bag 28 from, or a shelf for setting the effluent collection bag 28 on. In instances where the carriage assembly 22 is movable, a carriage assembly activation switch 30 may be provided with the drive unit 12, such as located on panel 16g to selectively position of the carriage assembly 22 inwardly or outwardly. Bag hooks 34 and 36 may extend through and/or from the panels 16e and 16f to hang saline bags and/or other fluid supply bags therefrom. The drive unit 12 may include a handle 42 as well as a plurality of wheels 52a-52n and brake pedals 54 for wheel lockage to assist in maneuvering the drive unit 12 by medical personnel.

In FIG. 1, the pump/catheter assembly 14 is shown detached from the drive unit 12. The pump/catheter assembly 14 includes an inflow pump 56 and a thrombectomy device 58. In some embodiments, the inflow pump 56 may be configured to provide fluid inflow through the thrombectomy device 58. During use, a portion of the pump/catheter assembly 14 may be secured within a portion of the drive unit 12. In some embodiments, the pump/catheter assembly 14 may include a bubble trap 60 attached to the inflow pump 56, a connection manifold assembly 62 connected to the bubble trap 60, a fixture 140, an effluent return tube 66 connected between the connection manifold assembly 62 and the thrombectomy device 58, a high-pressure fluid supply tube 64 attached between the output of the inflow pump 56 and the thrombectomy device 58 which may be coaxially arranged inside the effluent return tube 66, a transition fixture 69 between the distal end of the effluent return tube 66 and the proximal end of the thrombectomy device 58, an effluent waste tube 68 connecting the effluent collection bag 28 to the connection manifold assembly 62, and a fluid supply tube 70 having a bag spike 71 connecting a fluid supply bag 72 (e.g., a saline bag) to the connection manifold assembly 62. The fluid supply tube 70 may be in fluid communication with the interior of the bubble trap 60 to provide fluid from the fluid supply bag 72 to the inflow pump 56 and then to the thrombectomy device 58 through the high-pressure fluid supply tube 64.

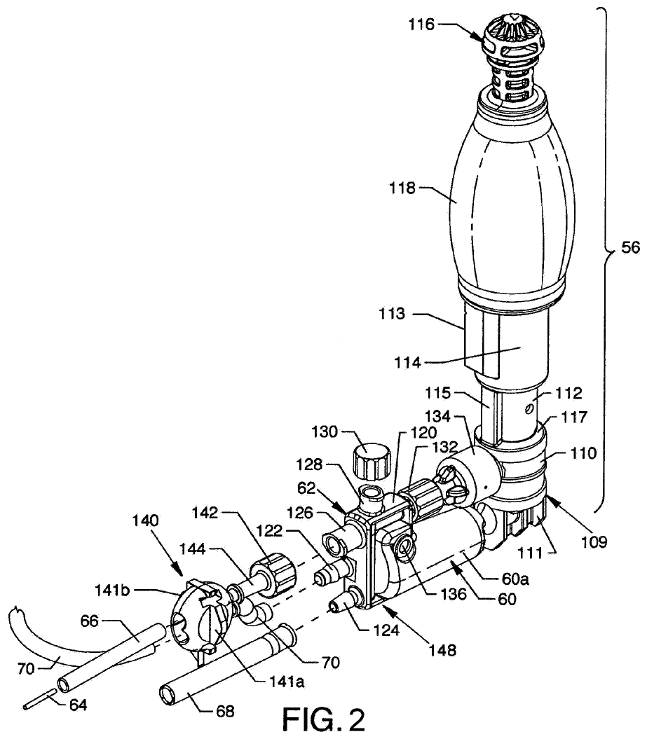

The drive unit 12 may include a reciprocating linear actuator 84 configured to engage a pump piston head 116 (e.g., FIG. 2) of the inflow pump 56 when the inflow pump 56 is engaged with the carriage assembly 22. The reciprocating linear actuator 84 may be disposed within the interior of the drive unit 12 and may be aligned with the pump piston head 116 (e.g., FIG. 2) when the inflow pump 56 is disposed within the interior of the drive unit 12. The reciprocating linear actuator 84 may be actuated such that reciprocating (e.g., up and down) strokes of the reciprocating linear actuator 84 drive the inflow pump 56 in response to activation of a user activation switch 40. In some embodiments, the user activation switch 40 may be a foot switch. In some embodiments, when the user activation switch 40 is depressed, the reciprocating linear actuator 84 and/or the inflow pump 56 is activated and/or runs, and when the user activation switch 40 is released, the reciprocating linear actuator 84 and/or the inflow pump 56 is stopped and/or ceases operation.

The drive unit 12 may include a display 31 including a user interface 32. In some embodiments, the user interface 32 may include memory and/or data storage capabilities. In some embodiments, the display 31 and/or the user interface 32 may be provided at the upper region of the drive unit 12 between the upper regions of the upper side panels 16e and 16f. In some embodiments, the display 31 and/or the user interface 32 may include a touchscreen user interface. Other configurations are also contemplated. Additional details associated with the display 31 and/or the user interface 32 are discussed below.

The drive unit 12 may include a controller 33 in electronic communication with the display 31, the user interface 32, the reciprocating linear actuator 84, the inflow pump 56, and/or the thrombectomy device 58. In some embodiments, the drive unit 12 and/or the controller 33 may include a data acquisition device 35. In some embodiments, the data acquisition device 35 may be disposed within the interior of the drive unit 12. In some embodiments, the data acquisition device 35 may be configured for wireless communication. Other configurations are also contemplated. In some embodiments, the user activation switch 40 may be in electronic communication with the drive unit 12 and/or the controller 33. In some embodiments, the user activation switch 40 may be in electronic communication with the drive unit 12 and/or the controller 33 via a wire or cable. In some embodiments, the user activation switch 40 may be in electronic communication with the drive unit 12 and/or the controller 33 wirelessly.

FIG. 2 is a partially exploded perspective view of several components of the pump/catheter assembly 14 (e.g., FIG. 1) generally including the inflow pump 56, the bubble trap 60, the connection manifold assembly 62, and the fixture 140. The inflow pump 56 centers about a tubular body 112. Components are located about the lower region of the tubular body 112 and include a base 109 having an upper portion 110 and a lower portion 111 both positioned about the lower region of the tubular body 112. An annular surface 117 is included at the top of the upper portion 110 of the base 109 for intimate contact with capture tabs of the carriage assembly 22 (e.g., FIG. 1) to retain the inflow pump 56 within and/or in engagement with the carriage assembly 22. A top body 114, is positioned about the upper region of the tubular body 112. The base 109 and the top body 114, as well as a connecting panel 115, may be molded or otherwise suitably constructed to encompass the greater part of the tubular body 112, for example.

In some embodiments, a data plate 113 may also be included on the pump/catheter assembly 14, such as on the top body 114 for example, for the inclusion of a barcode, an RFID tag, a data storage chip, informational displays, etc. to store, communicate, and/or otherwise determine specifications and/or operational parameters associated with the pump/catheter assembly 14, the thrombectomy device 58, the inflow pump 56, etc. and/or components thereof. In at least some embodiments, the data acquisition device 35 (e.g., FIG. 1) may be configured to communicate with the data plate 113 (e.g., the barcode, the RFID tag, the data storage chip, etc.), the pump/catheter assembly 14, the thrombectomy device 58, the inflow pump 56, etc. to obtain specifications and/or operational parameters associated with the pump/catheter assembly 14, the thrombectomy device 58, the inflow pump 56, etc. and/or components thereof. In some embodiments, the data acquisition device 35 (e.g., FIG. 1) may be configured to communicate with the data plate 113 (e.g., the barcode, the RFID tag, the data storage chip, etc.), the pump/catheter assembly 14, the thrombectomy device 58, the inflow pump 56, etc. to obtain identifying information related thereto which is associated with specifications and/or operational parameters stored in the memory of the user interface 32 and/or the controller 33. The identifying information may be used by the user interface 32 and/or the controller 33 to access the specifications and/or operational parameters associated with the pump/catheter assembly 14, the thrombectomy device 58, the inflow pump 56, etc. in use that is stored in the memory.

In some embodiments, the inflow pump 56 may include a hemispherically-shaped pump piston head 116 having a flexible boot 118 connected to and extending between the top body 114 and the pump piston head 116. In some instances, the lower portion 111 of the base 109 may serve as a mount for a first end of the bubble trap 60.

The connection manifold assembly 62 may be secured directly to a second end of the bubble trap 60 and in some instances may include a bracket 120 to which is attached a vertically oriented tubular manifold 148 having a plurality of ports attached or formed therethrough including a fluid (e.g., saline) inlet port 122, an effluent outlet port 124, a Luer style effluent return port 126, and/or an auxiliary port 128 and cap 130. Also shown are connectors 132 and 134 connectingly extending between the connection manifold assembly 62 and the upper portion 110 of the base 109.

The bubble trap 60 may include two mating halves of which a first bubble trap half 60a is shown. A hydrophobic filter 136 may be included at the upper forward region of the first bubble trap half 60a. In some embodiments, a second hydrophobic filter may be included on the second bubble trap half (not explicitly shown) which opposes the hydrophobic filter 136 on the first bubble trap half 60a.

The fixture 140, and components associated therewith, assists in support and connection of the effluent return tube 66 to the effluent return port 126 by a connector 142 combined continuously with a connection tube 144, and also assists in support, passage, and connection of the fluid supply tube 70 with the fluid inlet port 122. The fixture 140 may include outwardly extending vertically aligned and opposed tabs 141a and 141b which prevent the fixture 140 and associated effluent return tube 66 containing the high-pressure fluid supply tube 64 and the fluid supply tube 70 from contacting a roller pump (not shown) provided with the drive unit 12, such as located in the carriage assembly 22 (e.g., FIG. 1) or adjacent thereto.

In some embodiments, the effluent waste tube 68 may be positioned within and/or through the roller pump with the effluent collection bag 28 (e.g., FIG. 1) connected to the effluent waste tube 68. The effluent collection bag 28 may be suitably positioned for collecting effluent during the procedure. Pump rollers of the roller pump may engage the effluent waste tube 68 to control effluent fluid flow through the effluent waste tube 68 from the effluent outlet port 124 to the effluent collection bag 28. In some embodiments, other pump types and/or configurations may be used in place of the roller pump.

At an appropriate time, the thrombectomy device 58 may be subjected to a priming procedure to purge the thrombectomy device 58 of any air. For example, the tip of the thrombectomy device 58 may be placed in a bowl of sterile saline, or other fluid, and the inflow pump 56 may be operated by action of the reciprocating linear actuator 84 (such as by activating and/or depressing the user activation switch 40) to prime the thrombectomy device 58. Thereafter, medical personnel may insert the thrombectomy device 58 into the vasculature of the patient, and operation of the thrombectomy system 10 incorporating the user interface 32 and the user activation switch 40 can begin, as desired. The reciprocating linear actuator 84 is actuated according to the operating parameters to influence fluid inflow pressures, pump speed, flow rates, and the like to operate the inflow pump 56 to deliver pressurized fluid to the thrombectomy device 58 via the high-pressure fluid supply tube 64 residing in the effluent return tube 66. Supply fluid is routed through the bubble trap 60, may be pressurized by the inflow pump 56, and is routed through the high-pressure fluid supply tube 64 to the thrombectomy device 58 for use in a thrombectomy or other related procedure. Effluent may be returned through the effluent return tube 66 to the connection manifold assembly 62 for collection in the effluent collection bag 28 (e.g., FIG. 1) through the effluent waste tube 68, which may be controlled by the roller pump.

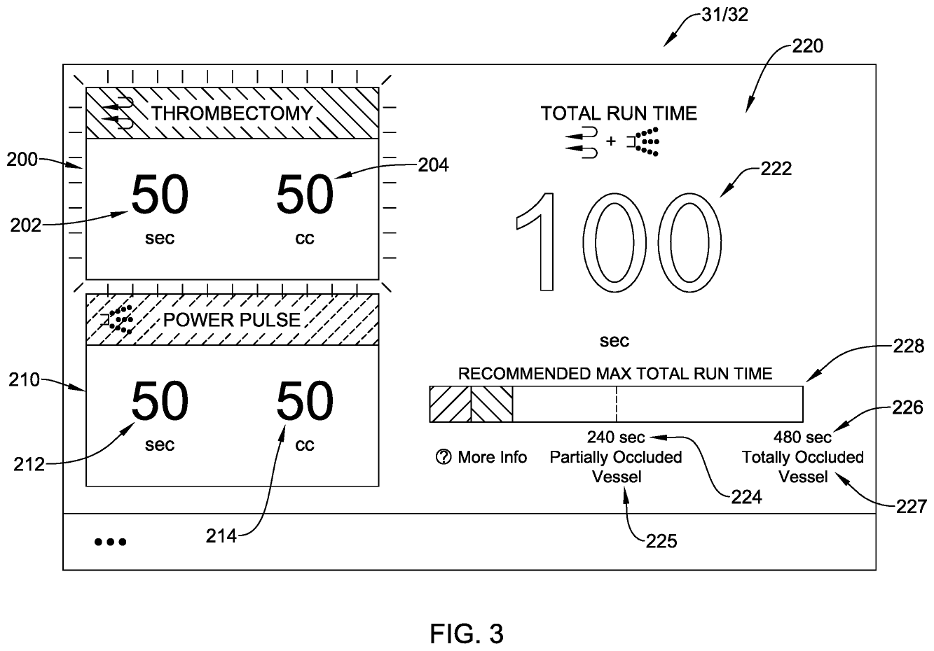

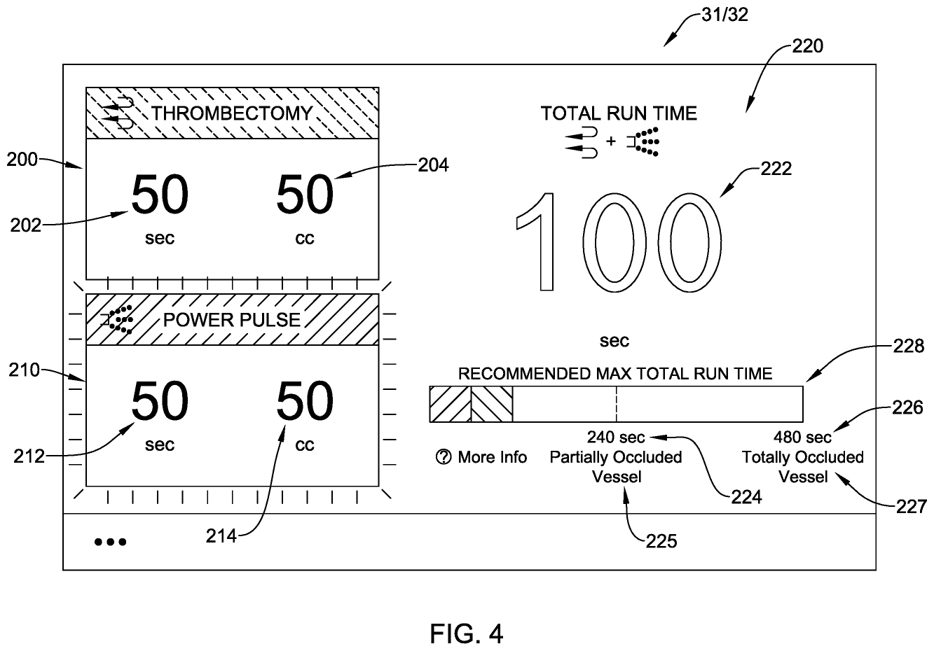

Turning now to FIGS. 3-4, selected aspects of the display 31 and/or the user interface 32 are illustrated. It is noted that the display 31 may be incorporated as part of the user interface 32 and/or the user interface 32 may be incorporated as part of the display 31. In other instances, the display 31 may be separate from the user interface 32. In some embodiments, the user interface 32/display 31 may include a first mode indicator 200 configured to indicate when a first mode of fluid inflow is active. In some embodiments, the first mode of fluid inflow may be a “thrombectomy” mode associated with ordinary use of a thrombectomy device, such as breaking up an occlusion or a clot. For example, in the first mode (e.g., “thrombectomy” mode), pressurized fluid may be advanced distally through the high-pressure fluid supply tube 64 toward the distal end of the thrombectomy device 56 by actuating the inflow pump 56. The pressurized fluid may be used to break up an occlusion or clot. In some embodiments, when the first mode of fluid inflow (e.g., “thrombectomy” mode) is active, the first mode indicator 200 may be colored, highlighted, lit up, flashing, or otherwise distinguished from other features on the display 31 and/or the user interface 32, as shown in FIG. 3 for example, to show that the first mode is active. For the purpose of illustration only, the color (or other distinguishing element) of the first mode indicator 200 and/or associated with the first mode of fluid inflow is shown using a first crosshatch that is angled from left to right in a downward direction. In some embodiments, the use of color as a distinguishing element may be preferred. In some embodiments, the first mode indicator 200 may be user selectable to activate the first mode of fluid inflow. In some embodiments, the first mode indicator 200 may be selectable via direct touch on a touchscreen of the user interface 32/display 31. In some embodiments, the first mode indicator 200 may be selectable via a button on the user interface 32. In some embodiments, the first mode indicator 200 may be selectable via the user activation switch 40. Other configurations are also contemplated. In FIG. 3, the first crosshatch is shown in solid lines to indicate that the first mode is active.

The “thrombectomy” mode may include fluid outflow (e.g., aspiration) from the treatment site to remove portions of the occlusion or the clot as it breaks up and/or to prevent infusion of excess fluid into the patient. In some embodiments, the first mode indicator 200 may include a first mode time display 202 showing an elapsed time that the first mode has been active and/or a first mode volume display 204 showing a volume of fluid infused through the thrombectomy system 10 and/or the thrombectomy device 58 in the first mode. While the values shown for the first mode time display 202 and the first mode volume display 204 in FIGS. 3-4 are the same (e.g., 50 sec (seconds) elapsed and 50 cc (milliliters) of fluid infused), indicating an infusion rate of 1 milliliter per second, these values are merely examples and different infusion rates may be used depending on the particular device in use, the fluid being infused, and/or selected patient parameters. For example, in some embodiments, the infusion rate may be within a range of about 0.35 milliliters per second to about 1.5 milliliters per second.

In some embodiments, the user interface 32 may include a second mode indicator 210 configured to indicate when a second mode of fluid inflow is active. In some embodiments, the second mode of fluid inflow may be a “Power Pulse™” mode associated with a particular use of a thrombectomy device. In some embodiments, the “Power Pulse™” mode may be used when an occlusion or a clot is calcified, more organized, and/or resistant to ordinary thrombectomy procedures. In some embodiments, when the second mode of fluid inflow (e.g., “Power Pulse™” mode) is active, the second mode indicator 210 may be colored, highlighted, lit up, flashing, or otherwise distinguished from other features on the display 31 and/or the user interface 32, as shown in FIG. 4 for example, to show that the second mode is active. For the purpose of illustration only, the color (or other distinguishing element) of the second mode indicator 210 and/or associated with the second mode of fluid inflow is shown using a second crosshatch that is angled from left to right in an upward direction. In some embodiments, the use of color as a distinguishing element may be preferred. In some embodiments, the second mode indicator 210 may be user selectable to activate the second mode of fluid inflow. In some embodiments, the second mode indicator 210 may be selectable via direct touch on a touchscreen of the user interface 32/display 31. In some embodiments, the second mode indicator 210 may be selectable via a button on the user interface 32. In some embodiments, the second mode indicator 210 may be selectable via the user activation switch 40. Other configurations are also contemplated. In FIG. 4, the second crosshatch is shown in solid lines to indicate that the second mode is active, and the first crosshatch is shown in phantom to indicate that the first mode is inactive.

In some embodiments, the “Power Pulse™” mode may include the introduction of a thrombolytic drug into the treatment site while limiting or preventing fluid outflow from the treatment site. In some embodiments, the “Power Pulse™” mode may be used to push or “force” the thrombolytic drug into the occlusion or clot to aid in breaking up the occlusion or clot. For example, in the second mode (e.g., “Power Pulse™” mode), pressurized fluid including a thrombolytic drug may be advanced distally through the high-pressure fluid supply tube 64 toward the distal end of the thrombectomy device 56 by actuating the inflow pump 56. The pressurized fluid including the thrombolytic drug may contact and/or penetrate into the occlusion or clot. In some procedures, the thrombolytic drug may be permitted to “dwell” at the treatment site to saturate into the occlusion or clot in order to soften and/or further break up the occlusion or clot before being removed (along with portions of the occlusion or clot) via fluid outflow (e.g., aspiration). For example, after running thrombectomy system 10 in the second mode (e.g., “Power Pulse™” mode) for a period of time (which runs the inflow pump 56 without running the roller pump of the effluent waste tube 68), the inflow pump 56 may be stopped so the thrombolytic drug may be permitted to “dwell” at the treatment site for a dwell time, (e.g., 1 minute or more, 2 minutes or more, 3 minutes or more, 4 minutes or more, 5 minutes or more, etc.), and thereafter the thrombectomy system 10 may be run in the first mode (e.g., thrombectomy mode) for an additional period of time (which runs the inflow pump 56 and the roller pump of the effluent waste tube 68) to break up and extract the occlusion or clot through the effluent waste tube 68 to the effluent collection bag 28. In some embodiments, the second mode indicator 210 may include a second mode time display 212 showing an elapsed time that the second mode has been active and/or a second mode volume display 214 showing a volume of fluid infused through the thrombectomy system 10 and/or the thrombectomy device 58 in the second mode. While the values shown for the second mode time display 212 and the second mode volume display 214 in FIGS. 3-4 are the same (e.g., 50 sec (seconds) elapsed and 50 cc (milliliters) of fluid infused), indicating an infusion rate of 1 milliliter per second, these values are merely examples and different infusion rates may be used depending on the particular device in use, the fluid being infused, and/or selected patient parameters. For example, in some embodiments, the infusion rate may be within a range of about 0.35 milliliters per second to about 1.5 milliliters per second.

In some embodiments, in the second mode, the thrombectomy system 10 may be configured to infuse, per an operator's control (e.g., “on demand”), single pulses of fluid at a time at about 0.62 milliliters per pulse. In some embodiments, in the second mode, the thrombectomy system 10 may be configured to infuse, per an operator's control (e.g., “on demand”), single pulses of fluid at a time within a range of about 0.50 milliliters per pulse to about 0.75 milliliters per pulse. Other configurations and/or ranges are also contemplated.

In some embodiments, the user interface 32/display 31 may include a run time display 220 including an alphanumeric run time value 222 shown in a first configuration. In some embodiments, the alphanumeric run time value 222 may be shown in the first configuration when fluid inflow is started and/or at a beginning of a procedure (e.g., when the alphanumeric run time value 222 equals or begins counting up from zero). In some embodiments, the first configuration may include a first color. In some embodiments, the first color may be white. In some embodiments, the first configuration may include a first size, a first brightness, and/or a first font style. Other configurations are also contemplated. The alphanumeric run time value 222 may be a sum of (e.g., may be cumulative of) a first run time value of the first mode of fluid inflow and a second run time value of the second mode of fluid inflow. The first run time value may be and/or may correspond to a first elapsed run time of the thrombectomy system 10 and/or the inflow pump 56 when activated (and/or when running or having run) in the first mode. The second run time value may be and/or may correspond to a second elapsed run time of the thrombectomy system 10 and/or the inflow pump 56 when activated (and/or running or having run) in the second mode. Accordingly, the alphanumeric run time value 222 may be and/or may correspond to a total elapsed run time of the thrombectomy system 10 and/or the inflow pump 56 when activated. In at least some embodiments, the alphanumeric run time value 222 may be displayed in seconds. Other units (e.g., minutes, minutes and seconds, etc.) are also contemplated. Additionally, in some embodiments, the alphanumeric run time value 222 may be and/or may correspond to a total elapsed run time of the thrombectomy system 10 across multiple thrombectomy devices if multiple thrombectomy devices are used on the same patient and/or within the same procedure. In some embodiments, the alphanumeric run time value 222 may be a sum of total elapsed run times for all thrombectomy devices used on the same patient and/or within the same procedure when multiple thrombectomy devices are used. For example, if three different thrombectomy devices are used, the alphanumeric run time value 222 may be a sum of the total elapsed run times for each of the three different thrombectomy devices (e.g., sum=device1totalelapsedruntime+device2totalelapsedruntime+device3totalelapsedruntime).

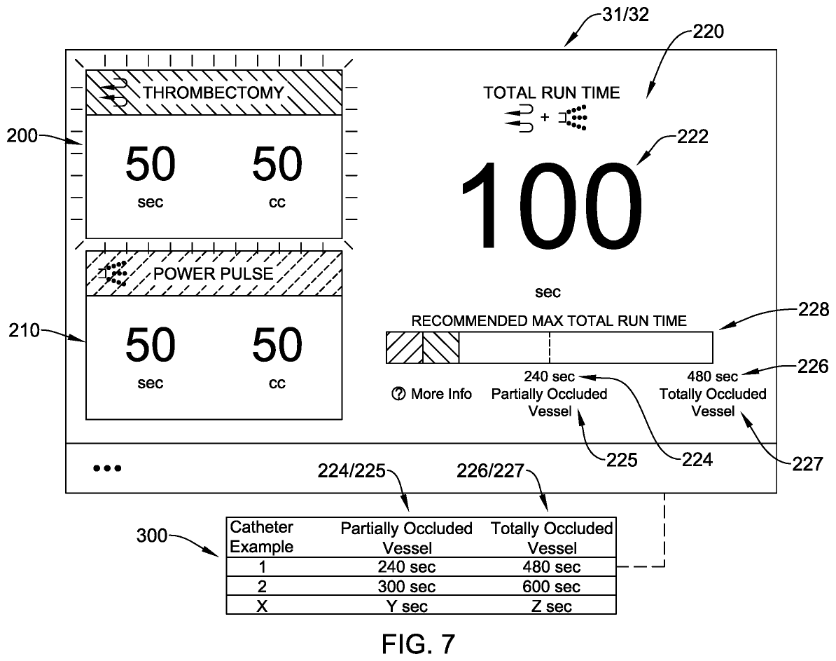

In some embodiments, the run time display 220 may include and/or may show a first recommended run time threshold 224 corresponding to a first working condition 225, such as a recommended run time threshold for treating a partially occluded vessel, and a second recommended run time threshold 226 corresponding to a second working condition 227, such as a recommended run time threshold for treating a totally occluded vessel. As discussed in more detail with respect to FIG. 7, the first recommended run time threshold 224 and the second recommended run time threshold 226 may be device specific. The second recommended run time threshold 226 is greater than the first recommended run time threshold 224. In at least some embodiments, the specifications and/or operational parameters associated with the pump/catheter assembly 14, the thrombectomy device 58, the inflow pump 56, etc. and/or components thereof may include the first recommended run time threshold 224 and the second recommended run time threshold 226.

In some embodiments, the user interface 32, the touchscreen user interface, and/or the run time display 220 may include a graphical display 228 (e.g., a bar graph, etc.) adjacent the alphanumeric run time value 222 showing the alphanumeric run time value 222 relative to the first recommended run time threshold 224 and the second recommended run time threshold 226. As seen in FIGS. 3-4, the graphical display 228 shows the alphanumeric run time value 222 as a sum (e.g., the total elapsed run time) of the first run time value (e.g., the first elapsed run time), shown illustratively using the first crosshatch, and the second run time value (e.g., the second elapsed run time), shown illustratively using the second crosshatch. As discussed above, the first crosshatch may be and/or represent the color of the first mode indicator 200 and/or associated with the first mode of fluid inflow and the second crosshatch may be and/or represent the color of the second mode indicator 210 and/or associated with the second mode of fluid inflow, which is different than the color of the first mode indicator 200 and/or associated with the first mode of fluid inflow.

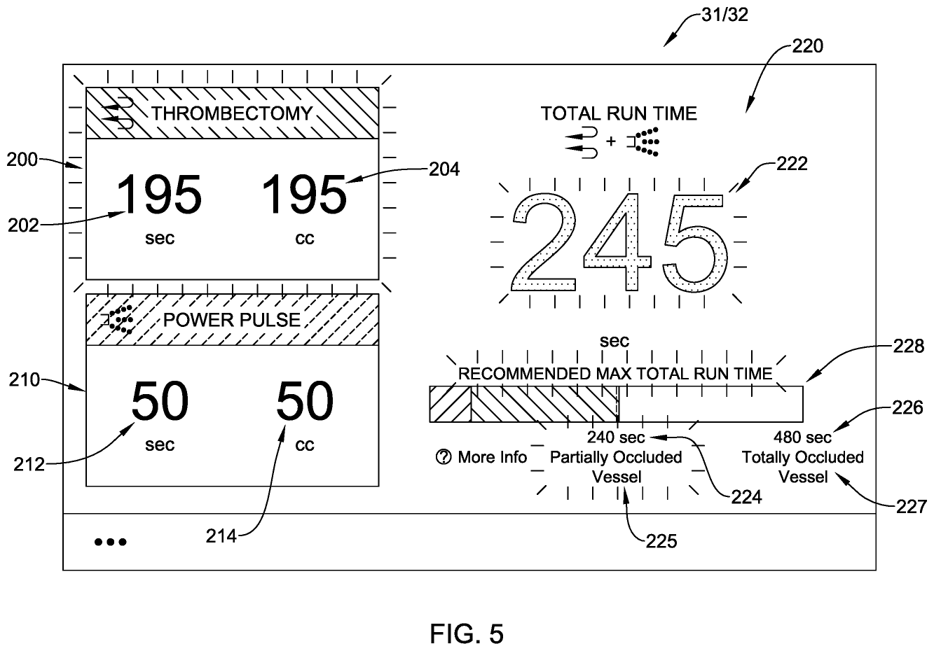

FIG. 5 illustrates selected aspects of the display 31 and/or the user interface 32 after the alphanumeric run time value 222 (e.g., the total elapsed run time) reaches and/or passes the first recommended run time threshold 224. In some embodiments, the user interface 32 and/or the controller 33 may be configured to provide a notification when the alphanumeric run time value 222 (e.g., the total elapsed run time) reaches and/or passes the first recommended run time threshold 224. In some embodiments, the alphanumeric run time value 222 may be configured to change from the first configuration (e.g., FIGS. 3-4) to a second configuration (e.g., FIG. 5) different from the first configuration when the sum (e.g., the total elapsed run time) of the first run time value (e.g., the first elapsed run time) and the second run time value (e.g., the second elapsed run time) reaches and/or passes the first recommended run time threshold 224. In some embodiments, the alphanumeric run time value 222 may be configured to change from the first color (e.g., FIGS. 3-4) to a second color (e.g., FIG. 5) different from the first color when the sum (e.g., the total elapsed run time) of the first run time value (e.g., the first elapsed run time) and the second run time value (e.g., the second elapsed run time) reaches and/or passes the first recommended run time threshold 224. In some embodiments, the second color may be yellow. Other colors and/or visual cues are also contemplated.

In some embodiments, the first recommended run time threshold 224 may be configured to change to the second color (e.g., FIG. 5) when the alphanumeric run time value 222 (e.g., the sum (e.g., the total elapsed run time) of the first run time value (e.g., the first elapsed run time) and the second run time value (e.g., the second elapsed run time)) reaches and/or passes the first recommended run time threshold 224.

In some embodiments, the user interface 32 and/or the controller 33 may be configured to emit a first audible notification (e.g., a first “chime”) when the alphanumeric run time value 222 (e.g., the total elapsed run time) reaches and/or passes the first recommended run time threshold 224. In some embodiments, a volume of the first audible notification may be user selectable and/or user defined before starting the procedure. In some embodiments, a tone, melody, or sound sequence of the first audible notification may be user selectable and/or user defined before starting the procedure.

After the alphanumeric run time value 222 (e.g., the total elapsed run time) reaches and/or passes the first recommended run time threshold 224, the thrombectomy system 10, the reciprocating linear actuator 84, and/or the inflow pump 56 may continue to run whenever the user activation switch 40 is activated. As such, the alphanumeric run time value 222 (e.g., the total elapsed run time) reaching or passing the first recommended run time threshold 224 does not cause the thrombectomy system 10, the reciprocating linear actuator 84, and/or the inflow pump 56 to shut down or stop providing fluid inflow and/or fluid outflow whenever the user activation switch 40 is activated. The first audible notification and/or the second color may serve as a first notification and/or reminder for the user to be conscious and/or cautious of the amount of fluid that has been infused into the patient. Each procedure and/or patient may be different and different amounts of fluid may be infused based on various conditions and/or parameters. For example, some factors and/or conditions that may be considered when deciding if and/or how long to use the thrombectomy system 10 may include the state of the patient's kidneys, the amount of contrast used, the size of the patient, the state of hydration of the patient, and/or the technique being used with the thrombectomy system 10. This list should not be considered all-inclusive, and other factors and/or conditions may also be relevant to a particular procedure.

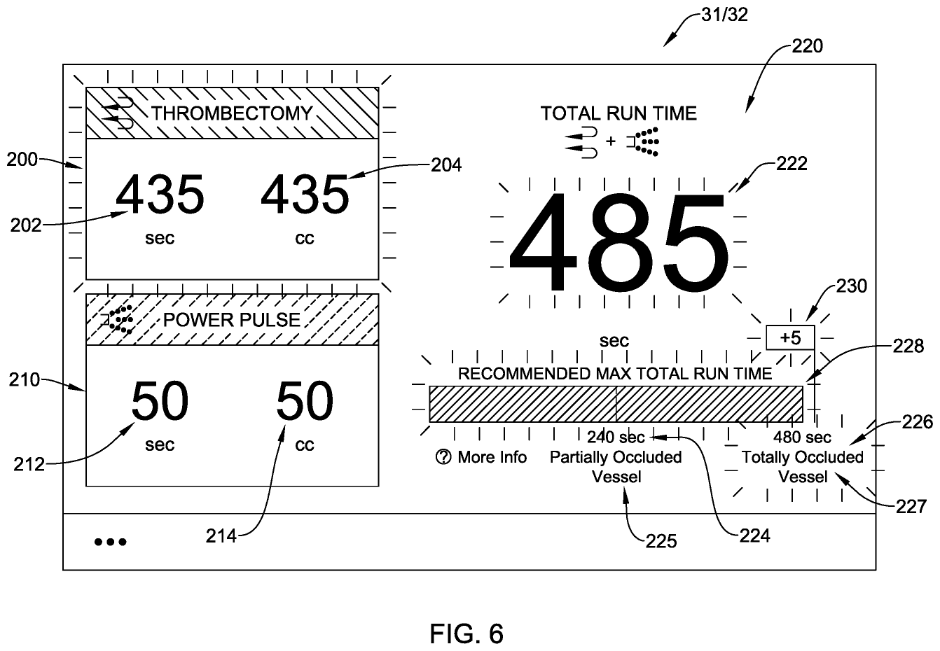

FIG. 6 illustrates selected aspects of the display 31 and/or the user interface 32 after the alphanumeric run time value 222 (e.g., the total elapsed run time) reaches and/or passes the second recommended run time threshold 226. In some embodiments, the user interface 32 and/or the controller 33 may be configured to provide a second notification when the alphanumeric run time value 222 (e.g., the total elapsed run time) reaches and/or passes the second recommended run time threshold 226. In some embodiments, the alphanumeric run time value 222 may be configured to change to a third configuration (e.g., FIG. 6) different from the first configuration and the second configuration when the sum (e.g., the total elapsed run time) of the first run time value (e.g., the first elapsed run time) and the second run time value (e.g., the second elapsed run time) reaches and/or passes the second recommended run time threshold 226. In some embodiments, the alphanumeric run time value 222 may be configured to change to a third color (e.g., FIG. 6) different from the first color and the second color when the sum (e.g., the total elapsed run time) of the first run time value (e.g., the first elapsed run time) and the second run time value (e.g., the second elapsed run time) reaches and/or passes the second recommended run time threshold 226. In some embodiments, the third color may be orange. Other colors and/or visual cues are also contemplated.

In some embodiments, the second recommended run time threshold 226 may be configured to change to the third color (e.g., FIG. 6) when the alphanumeric run time value 222 (e.g., the sum (e.g., the total elapsed run time) of the first run time value (e.g., the first elapsed run time) and the second run time value (e.g., the second elapsed run time)) reaches and/or passes the second recommended run time threshold 226.

In some embodiments, the user interface 32 and/or the controller 33 may be configured to emit a second audible notification (e.g., a second “chime”) when the alphanumeric run time value 222 (e.g., the total elapsed run time) reaches and/or passes the second recommended run time threshold 226. In some embodiments, a volume of the second audible notification may be user selectable and/or user defined before starting the procedure. In some embodiments, a tone, melody, or sound sequence of the second audible notification may be user selectable and/or user defined before starting the procedure. In some embodiments, the second audible notification (e.g., the second “chime”) may be the first audible notification (e.g., the first “chime”) emitted twice in rapid succession.

In some embodiments, after the alphanumeric run time value 222 (e.g., the total elapsed run time) reaches and/or passes the second recommended run time threshold 226, the graphical display 228 may be configured to change to the third color (e.g., FIG. 6). For the purpose of illustration only, the third color (or other distinguishing element) of the graphical display 228 in FIG. 6 is shown using a third crosshatch that is angled from left to right in an upward direction at a higher density than the second crosshatch. In some embodiments, the use of color as a distinguishing element may be preferred.

It is noted that in some embodiments, the color of the alphanumeric run time value 222 may not change when the sum (e.g., the total elapsed run time) of the first run time value (e.g., the first elapsed run time) and the second run time value (e.g., the second elapsed run time) reaches and/or passes the first recommended run time threshold 224. Rather, only a first audible notification may be emitted and/or a banner or notification may be momentarily displayed on the display 31 and or user interface 32, for example.

In some instances, the color of the alphanumeric run time value 222 may only change after the alphanumeric run time value 222 (e.g., the total elapsed run time) reaches and/or passes the second recommended run time threshold 226, at which point the graphical display 228 may be configured to change to a second color.

In such instances, the user interface 32 and/or the controller 33 may be configured to emit a second audible notification (e.g., a second “chime”) when the alphanumeric run time value 222 (e.g., the total elapsed run time) reaches and/or passes the second recommended run time threshold 226. In some embodiments, a volume of the second audible notification may be user selectable and/or user defined before starting the procedure. In some embodiments, a tone, melody, or sound sequence of the second audible notification may be user selectable and/or user defined before starting the procedure. In some embodiments, the second audible notification (e.g., the second “chime”) may be the first audible notification (e.g., the first “chime”) emitted twice in rapid succession.

After the alphanumeric run time value 222 (e.g., the total elapsed run time) reaches and/or passes the second recommended run time threshold 226, the thrombectomy system 10, the reciprocating linear actuator 84, and/or the inflow pump 56 may continue to run whenever the user activation switch 40 is activated. As such, the alphanumeric run time value 222 (e.g., the total elapsed run time) reaching or passing the second recommended run time threshold 226 does not cause the thrombectomy system 10, the reciprocating linear actuator 84, and/or the inflow pump 56 to shut down or stop providing fluid inflow and/or fluid outflow whenever the user activation switch 40 is activated. The second audible notification and/or the third color may serve as a second notification and/or reminder for the user to be more conscious and/or more cautious of the amount of fluid that has been infused into the patient than at the first audible notification and/or the second color. Each procedure and/or patient may be different and different amounts of fluid may be infused based on various conditions and/or parameters. As discussed above, some factors and/or conditions that may be considered when deciding if and/or how long to use the thrombectomy system 10 may include the state of the patient's kidneys, the amount of contrast used, the size of the patient, the state of hydration of the patient, and/or the technique being used with the thrombectomy system 10. This list should not be considered all-inclusive, and other factors and/or conditions may also be relevant to a particular procedure.

In some embodiments, after the alphanumeric run time value 222 (e.g., the total elapsed run time) reaches and/or passes the second recommended run time threshold 226, the user interface 32 may include and/or may initiate a flag timer 230 that counts up to show how long the thrombectomy system 10, the reciprocating linear actuator 84, and/or the inflow pump 56 has continued to run past the second recommended run time threshold 226 whenever the user activation switch 40 is activated. For example, if the second recommended run time threshold 226 is 480 sec (seconds), and the alphanumeric run time value 222 (e.g., the total elapsed run time) is 485 sec (seconds), the flag timer 230 may read +5 (indicating that the total run time is 5 seconds longer than the second recommended run time threshold 226), as shown in FIG. 6. Thus, the user interface 32/display 31 may display the flag timer 230 as the amount of time the thrombectomy system 10 has been running beyond the second recommended run time threshold 226. This information may provide an additional reference point to the user when considering how much fluid has been infused into the patient with respect to actual conditions and/or patient parameters. In at least some embodiments, the flag timer 230 may be shown in the third color, thereby indicating it is related to and/or associated with reaching and/or passing the second recommended run time threshold 226.

In some embodiments, after reaching the first recommended run time threshold 224, no change may occur on the user interface 32 and/or the display 31. As such, in some embodiments, the user interface 32 and/or the controller 33 may be configured to provide a notification when the alphanumeric run time value 222 (e.g., the total elapsed run time) reaches and/or passes the second recommended run time threshold 226. In some embodiments, the alphanumeric run time value 222 may be configured to change from the first configuration (e.g., FIGS. 3-4) to a second configuration (e.g., FIG. 5, FIG. 6) different from the first configuration when the sum (e.g., the total elapsed run time) of the first run time value (e.g., the first elapsed run time) and the second run time value (e.g., the second elapsed run time) reaches and/or passes the second recommended run time threshold 226. In some embodiments, the alphanumeric run time value 222 may be configured to change from the first color (e.g., FIGS. 3-4) to a second color (e.g., FIG. 5, FIG. 6) different from the first color when the sum (e.g., the total elapsed run time) of the first run time value (e.g., the first elapsed run time) and the second run time value (e.g., the second elapsed run time) reaches and/or passes the second recommended run time threshold 226. In some embodiments, the second color may be yellow. In some embodiments, the second color may be orange. Other colors and/or visual cues are also contemplated.

In some embodiments, the second recommended run time threshold 226 may be configured to change to the second color (e.g., FIG. 5, FIG. 6) when the alphanumeric run time value 222 (e.g., the sum (e.g., the total elapsed run time) of the first run time value (e.g., the first elapsed run time) and the second run time value (e.g., the second elapsed run time)) reaches and/or passes the second recommended run time threshold 226.

In some embodiments, the user interface 32 and/or the controller 33 may be configured to emit a first audible notification (e.g., a first “chime”) when the alphanumeric run time value 222 (e.g., the total elapsed run time) reaches and/or passes the second recommended run time threshold 226. In some embodiments, a volume of the first audible notification may be user selectable and/or user defined before starting the procedure. In some embodiments, a tone, melody, or sound sequence of the first audible notification may be user selectable and/or user defined before starting the procedure.

In some embodiments, the alphanumeric run time value 222 (e.g., the total elapsed run time) reaching and/or passing the second recommended run time threshold 226 may be the first instance, time, or occasion where any change in color occurs on the user interface 32 and/or the display 31, and/or may be the first instance, time, or occasion where any audible notification (e.g., “chime”) is emitted.

After the alphanumeric run time value 222 (e.g., the total elapsed run time) reaches and/or passes the second recommended run time threshold 226, the thrombectomy system 10, the reciprocating linear actuator 84, and/or the inflow pump 56 may continue to run whenever the user activation switch 40 is activated. As such, the alphanumeric run time value 222 (e.g., the total elapsed run time) reaching or passing the second recommended run time threshold 226 does not cause the thrombectomy system 10, the reciprocating linear actuator 84, and/or the inflow pump 56 to shut down or stop providing fluid inflow and/or fluid outflow whenever the user activation switch 40 is activated. The first audible notification and/or the second color may serve as a notification and/or reminder for the user to be conscious and/or cautious of the amount of fluid that has been infused into the patient. Each procedure and/or patient may be different and different amounts of fluid may be infused based on various conditions and/or parameters. For example, some factors and/or conditions that may be considered when deciding if and/or how long to use the thrombectomy system 10 may include the state of the patient's kidneys, the amount of contrast used, the size of the patient, the state of hydration of the patient, and/or the technique being used with the thrombectomy system 10. This list should not be considered all-inclusive, and other factors and/or conditions may also be relevant to a particular procedure.

In some embodiments, after the alphanumeric run time value 222 (e.g., the total elapsed run time) reaches and/or passes a recommended run time threshold, such as the second recommended run time threshold 226, the user interface 32 may include and/or may initiate a flag timer 230 that counts up to show how long the thrombectomy system 10, the reciprocating linear actuator 84, and/or the inflow pump 56 has continued to run past the recommended run time threshold, such as the second recommended run time threshold 226, whenever the user activation switch 40 is activated. For example, if the second recommended run time threshold 226 is 480 sec (seconds), and the alphanumeric run time value 222 (e.g., the total elapsed run time) is 485 sec (seconds), the flag timer 230 may read +5 (indicating that the total run time is 5 seconds longer than the second recommended run time threshold 226), as shown in FIG. 6. Thus, the user interface 32/display 31 may display the flag timer 230 as the amount of time the thrombectomy system 10 has been running beyond a recommended run time threshold, such as the second recommended run time threshold 226. This information may provide an additional reference point to the user when considering how much fluid has been infused into the patient with respect to actual conditions and/or patient parameters. In at least some embodiments, the flag timer 230 may be shown in the second color, thereby indicating it is related to and/or associated with reaching and/or passing the recommended run time threshold, such as the second recommended run time threshold 226. FIG. 7 illustrates selected aspects of the display 31 and/or the user interface 32, as well as a look-up table 300. In some embodiments, the look-up table 300 may be included in and/or stored in the memory of the user interface 32 and/or the controller 33 (e.g., FIG. 1). The look-up table 300 may include specifications and/or operational parameters associated with the pump/catheter assembly 14, the thrombectomy device 58, the inflow pump 56, etc. The specifications and/or operational parameters associated with the pump/catheter assembly 14, the thrombectomy device 58, the inflow pump 56, etc. may be associated with identifying information associated with pump/catheter assembly 14, the thrombectomy device 58, the inflow pump 56, etc.

For example, the look-up table 300 may include specifications and/or operational parameters for a plurality of devices, shown in FIG. 7 as “catheter example” 1, 2, X, etc. The specifications and/or operational parameters may include a first recommended run time threshold 224 associated with a first working condition 225 (shown as “fully occluded vessel” in look-up table 300) and a second recommended run time threshold 226 associated with a second working condition 227 (shown as “fully occluded vessel” in look-up table 300).

In the examples shown, catheter example 1 has a first recommended run time threshold 224 associated with a first working condition 225 (shown as “partially occluded vessel” in the look-up table 300) of 240 sec (seconds) and a second recommended run time threshold 226 associated with a second working condition 227 (shown as “fully occluded vessel” in look-up table 300) of 480 sec (seconds). In the examples shown, catheter example 2 has a first recommended run time threshold 224 associated with a first working condition 225 (shown as “partially occluded vessel” in the look-up table 300) of 300 sec (seconds) and a second recommended run time threshold 226 associated with a second working condition 227 (shown as “fully occluded vessel” in look-up table 300) of 600 sec (seconds).

Accordingly, additional examples X may be included in the look-up table 300 with a first recommended run time threshold 224 associated with a first working condition 225 (shown as “partially occluded vessel” in the look-up table 300) of Y sec (seconds) and a second recommended run time threshold 226 associated with a second working condition 227 (shown as “fully occluded vessel” in look-up table 300) of Z sec (seconds). In general, the second recommended run time threshold 226 may be about two times the first recommended run time threshold 224. However, other values and/or ratios are also contemplated. In one non-limiting example, a catheter example X1 may have a second recommended run time threshold 226 that is about 1.5 times the first recommended run time threshold 224. In another non-limiting example, a catheter example X2 may have a second recommended run time threshold 226 that is about 2.5 times the first recommended run time threshold 224.

In other instances, the first recommended run time threshold 224 associated with a first working condition 225 (e.g., treatment of a partially occluded vessel) and the second recommended run time threshold 226 associated with a second working condition 227 (e.g., treatment of a fully occluded vessel) may be read from an RFID tag provided with the particular catheter selected for a given medical procedure and provided to the controller 33 of the drive unit 12.

The controller 33 and/or the user interface 32 may be configured to provide the specifications and/or operational parameters (which may include the first recommended run time threshold 224 associated with a first working condition 225 and the second recommended run time threshold 226 associated with a second working condition 227 associated with the pump/catheter assembly 14, the thrombectomy device 58, the inflow pump 56, etc.) to the run time display 220 based on the identifying information obtained by the data acquisition device 35 (e.g., FIG. 1) from the data plate 113, for example.

The materials that can be used for the various components of the thrombectomy system and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the drive unit 12, the pump/catheter assembly 14, the inflow pump 56, the thrombectomy device 58, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A user interface for a thrombectomy fluid management system, comprising:
a first mode indicator configured to indicate when a first mode of fluid inflow is active;
a second mode indicator configured to indicate when a second mode of fluid inflow is active; and
a run time display including an alphanumeric run time value shown in a first color;
wherein the alphanumeric run time value is a sum of a first run time value of the first mode and a second run time value of the second mode.

2. The user interface of claim 1, wherein the run time display shows a first recommended run time threshold corresponding to a first working condition and a second recommended run time threshold corresponding to a second working condition.

3. The user interface of claim 2, wherein the alphanumeric run time value is configured to change from the first color to a second color when the sum reaches at least one of the first and second recommended run time thresholds.

4. The user interface of claim 2, wherein the alphanumeric run time value is configured to change from the first color to a second color when the sum reaches the first recommended run time threshold, and the alphanumeric run time value is configured to change to a third color when the sum reaches the second recommended run time threshold.

5. The user interface of claim 1, wherein the first mode indicator is user selectable to activate the first mode of fluid inflow.

6. The user interface of claim 1, wherein the second mode indicator is user selectable to activate the second mode of fluid inflow.

7. The user interface of claim 1, wherein the alphanumeric run time value is shown in seconds.

8. A thrombectomy fluid management system, comprising:
an inflow pump configured to provide fluid inflow through a thrombectomy device;
a controller in communication with the inflow pump and the thrombectomy device;
a user activation switch configured to activate the inflow pump; and
a display including a user interface;
wherein the user interface includes:
a first mode indicator configured to indicate when a first mode of fluid inflow is active;
a second mode indicator configured to indicate when a second mode of fluid inflow is active; and
a run time display including an alphanumeric run time value shown in a first color;
wherein the alphanumeric run time value is a sum of a first run time value of the first mode and a second run time value of the second mode.

9. The thrombectomy fluid management system of claim 8, wherein the run time display shows a first recommended run time threshold corresponding to a first working condition and a second recommended run time threshold corresponding to a second working condition.

10. The thrombectomy fluid management system of claim 9, wherein the alphanumeric run time value is configured to change from the first color to a second color when the sum reaches at least one of the first and second recommended run time thresholds.

11. The thrombectomy fluid management system of claim 10, wherein the controller is configured to emit a first audible notification when the sum reaches the first recommended run time threshold.

12. The thrombectomy fluid management system of claim 9, wherein the alphanumeric run time value is configured to change from the first color to a second color when the sum reaches the first recommended run time threshold, and the alphanumeric run time value is configured to change to a third color when the sum reaches the second recommended run time threshold.

13. The thrombectomy fluid management system of claim 12, wherein the controller is configured to emit a second audible notification when the sum reaches the second recommended run time threshold.

14. The thrombectomy fluid management system of claim 9, wherein after reaching the first recommended run time threshold, the inflow pump continues to run whenever the user activation switch is activated.

15. The thrombectomy fluid management system of claim 9, wherein after reaching the second recommended run time threshold, the inflow pump continues to run whenever the user activation switch is activated.

16. A thrombectomy fluid management system, comprising:
an inflow pump configured to provide fluid inflow through a thrombectomy device;
a controller in communication with the inflow pump and the thrombectomy device;
a user activation switch configured to activate the inflow pump; and
a display including a touchscreen user interface;
wherein the touchscreen user interface includes:
a first mode indicator configured to highlight when a first mode of fluid inflow is active, the first mode indicator being user selectable on the touchscreen user interface;
a second mode indicator configured to highlight when a second mode of fluid inflow is active, the second mode indicator being user selectable on the touchscreen user interface; and
a run time display including an alphanumeric run time value shown in a first color when fluid inflow is started;
wherein the alphanumeric run time value is a sum of a first elapsed run time of the inflow pump when activated in the first mode and a second elapsed run time of the inflow pump when activated in the second mode.

17. The thrombectomy fluid management system of claim 16, wherein the run time display shows a first recommended run time threshold corresponding to a first working condition and a second recommended run time threshold corresponding to a second working condition.

18. The thrombectomy fluid management system of claim 17, wherein the controller includes data acquisition device configured to communicate with the thrombectomy device to obtain specifications associated with the thrombectomy device.

19. The thrombectomy fluid management system of claim 18, wherein the specifications include the first recommended run time threshold and the second recommended run time threshold.

20. The thrombectomy fluid management system of claim 17, wherein the touchscreen user interface includes a graphical display adjacent the alphanumeric run time value showing the alphanumeric run time value relative to the first recommended run time threshold and the second recommended run time threshold.
